(19) United States
(12) Patent Application Publication
(10) Pub. No.: US 2026/0216429 A1
(43) Pub. Date: Jul. 30, 2026

(54) INFUSION SYSTEM

(71) Applicant: SIR RUN RUN SHAW HOSPITAL OF ZHEJIANG UNIVERSITY, Zhejiang (CN)

(72) Inventors: Xin YU, Zhejiang (CN); Hong YU, Zhejiang (CN); Hongyuan LV, Zhejiang (CN); Bo SHEN, Zhejiang (CN); Ying CHENG, Zhejiang (CN)

(73) Assignee: SIR RUN RUN SHAW HOSPITAL OF ZHEJIANG UNIVERSITY, Zhejiang (CN)

(21) Appl. No.: 18/722,503

(22) PCT Filed: Oct. 20, 2023
(86) PCT No.: PCT/CN2023/125518

§ 371 Date: Jun. 20, 2024

(30) Foreign Application Priority Data
Aug. 10, 2023 (CN) 202311008614.X

(51) Int. Cl. A61M 5/168; A61M 39/22

(52) U.S. Cl. CPC A61M 5/16854; A61M 39/223; A61M 2205/18; A61M 2205/3334; A61M 2205/6009

(57) ABSTRACT

An infusion system is provided and includes: a three-way valve, provided with a first liquid inlet for an intravenous infusion liquid, a second liquid inlet configured for a syringe liquid and a liquid outlet communicating with a venipuncture needle; an information matching structure, including a first identification code configured for identifying identity information of the three-way valve and an identifier configured for identifying the first identification code; a detection structure, including a first flow sensor configured for detecting a liquid flow rate at the first liquid inlet, a second flow sensor configured for detecting a liquid flow rate at the liquid outlet and a pressure sensor configured for detecting a pressure at the liquid outlet; and a controller, is capable of acquiring identity information, flow and pressure information, recording the information to corresponding identity information items and giving an alarm when the information is abnormal.

### CROSS-REFERENCE TO RELATED APPLICATION

This application is a national stage application of International Patent Application No. PCT/CN 2023/125518, filed on Oct. 20, 2023, which claims the priority of Chinese Patent Application No. 202311008614.X entitled “INFUSION SYSTEM” filed with the Chinese Patent Office on Aug. 10, 2023, which is incorporated herein by reference in its entirety.

### TECHNICAL FIELD

The present disclosure belongs to the field of medical devices, and in particular to an infusion system.

### BACKGROUND

Opioids are alkaloids extracted from opium poppy and their derivatives in vivo and in vitro, which can interact with central specific receptors to relieve pain, mainly include analgesic drugs such as morphine, codeine, dihydrocodeine, hydromorphone, oxycodone, fentanyl, sufentanil, remifentanil and pethidine, and are often used for moderate and severe pain treatment and perioperative anesthesia of patients. However, the use of opioids in large doses may lead to stupor, coma and respiratory depression, and repeated use may cause the changes of tolerance and neurological addiction of the body, leading to physiological, psychological and social harm. Therefore, as a controlled drug, the use of the opioids in clinical practice must be strictly controlled, and the dosage, purpose and situation of use must be accurately grasped.

Anesthesiology department is the clinical department with the largest use of opioids. At present, the routine anesthesiology department receives the corresponding number of opioids from the drug administrator according to the surgical amount and the physician order of the surgery on that day, and after the surgery on that day, the remaining drugs and empty drug bottles are returned, and individual registration and medication records are made. However, this method of manually managing drugs is not conducive to the fine control of the amount of the in and out of opioids, but also causes the waste of redundant drugs.

### SUMMARY

The embodiments aim to overcome disadvantages in the prior art, an infusion system is provided, aiming at achieving accurate control of drugs.

An infusion system includes:
a three-way valve, provided with a first liquid inlet for an intravenous infusion liquid, a second liquid inlet for a syringe liquid and a liquid outlet communicating with a venipuncture needle;
an information matching structure, including a first identification code for identifying identity information of the three-way valve and an identifier for identifying the first identification code;
a detection structure, including a first flow sensor for detecting a liquid flow rate at the first liquid inlet, a second flow sensor for detecting a liquid flow rate at the liquid outlet and a pressure sensor for detecting a pressure at the liquid outlet; and
a controller, capable of to acquiring the identity information identified by the identifier and acquiring information detected by the first flow sensor, the second flow sensor and the pressure sensor, correspondingly recording the information to corresponding identity information items, and controlling a warning indicator to give an alarm when the information is abnormal.

Alternatively, the warning indicator is a voice warning indicator arranged at the three-way valve.

Alternatively, the three-way valve is provided with an attachment area, the attachment area is configured for attaching a second identification code, and the second identification code is configured for identifying patient information. The identifier can identify the second identification code.

Alternatively, the controller is configured to send successful matching information after receiving the identity information and the patient information.

Alternatively, the controller is configured to acquire and record the information detected by the first flow sensor and the second flow sensor, including injection time, injection dosage and stop time.

Alternatively, the controller, the controller is configured to control the warning indicator to give the alarm when the controller receives pressure information of the liquid outlet but not receive flow information of the first liquid inlet or flow information of the second liquid inlet, or when the controller receives the flow information of the first liquid inlet or the flow information of the second liquid inlet but not receive the pressure information of the liquid outlet.

Alternatively, the controller is configured to control the warning indicator to give the alarm when a pressure at the liquid outlet is greater than a preset threshold.

Alternatively, the controller is configured to send system start information when flow information of the first liquid inlet or flow information of the second liquid inlet is matched with the pressure information of the liquid outlet.

Alternatively, the detection structure further includes a second pressure sensor at the second liquid inlet. The second pressure sensor sends a pressure signal when the syringe is connected with the second liquid inlet, and the controller sends successful connection information when the controller receives the pressure signal.

Alternatively, the three-way valve is provided with a reader for identifying drug information of the syringe at the second liquid inlet.

Alternatively, the drug information includes a drug name and a drug concentration.

The infusion system provided by the embodiments can accurately control the drug dosage through the detection structure and the information matching structure, thus facilitating the accurate control of the amount of the in and out of the drugs, and solving the problem of drug waste.

### BRIEF DESCRIPTION OF THE DRAWINGS

To describe the technical solutions of the present disclosure more clearly, the following briefly introduces the drawings required for describing the embodiments. Apparently, the drawings in the following description show merely some embodiments of the present disclosure, and those of ordinary skill in the art may still derive other drawings from these drawings without creative efforts.

FIG. 1 is a structural schematic diagram of a three-way valve according to an embodiment of the present disclosure;

FIG. 2 is a schematic diagram of a three-way valve according to the embodiment of the present disclosure from another angle.

### LIST OF REFERENCE CHARACTERS

10 three way valve; 11 first liquid inlet; 12 second liquid inlet; 21 liquid outlet; 18 first flow sensor; 17 second flow sensor; 14 attachment area; 141 second identification code; 15 warning indicator; 16 reader; 19 pressure sensor; 13 rotary switch; 20 chip.

### DETAILED DESCRIPTION OF THE EMBODIMENTS

In order to make the objectives, technical solutions and advantages of the present disclosure more clearly, the present disclosure is further described in detail below with reference to the drawings and embodiments. It should be understood that specific embodiments described here are only used to illustrate rather than limiting the present disclosure.

It should be noted that when an element is described “fixed” or “disposed” on another element, it may be directly or indirectly on another element. It should be noted that when an element is described as “connected” to another element, it may be directly or indirectly connected with another element.

It also should be noted that the orientation terms such as left, right, up and down in the embodiment of the present disclosure are only relative concepts or reference to the normal use state of the product, and should not be considered as restrictive.

An infusion system is provided by this embodiment, including a three-way valve 10, an information matching structure, a detection structure, a controller and a warning indicator 15.

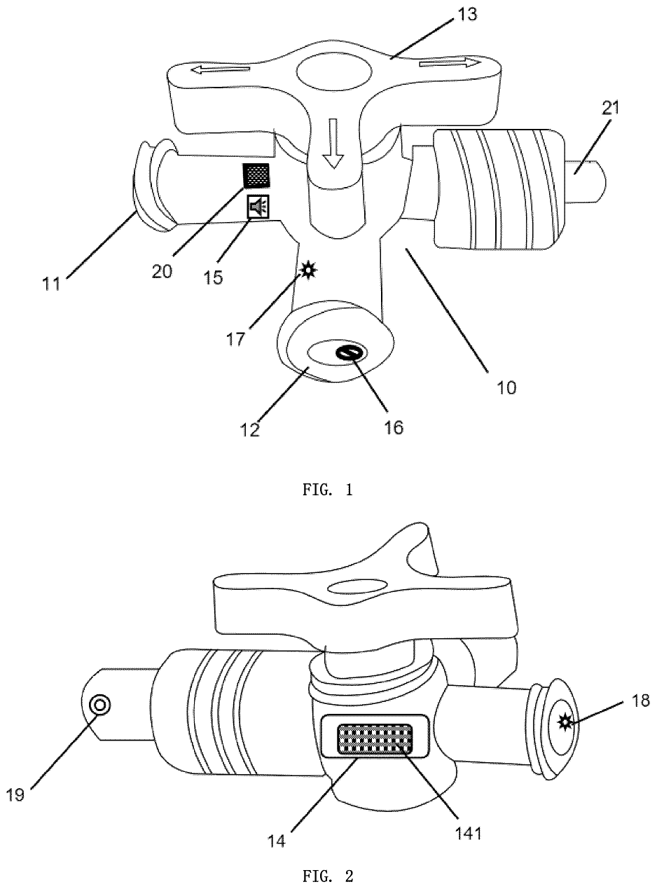

Please referring to FIG. 1 and FIG. 2, the three-way valve 10 includes a valve body and a rotary switch 13. The valve body is provided with two liquid inlets (correspondingly to a first liquid inlet 11 and a second liquid inlet 12) and a liquid outlet 21. The rotary switch 13 is configured to adjust a connection relationship between the two liquid inlets and the liquid outlet 21. Specifically, the rotary switch 13 is connected with a valve core in the valve body, the valve core is located at an intersection of the two liquid inlets (correspondingly to the first liquid inlet 11 and the second liquid inlet 12) and the liquid outlet 21, and the rotation of the valve core is achieved by rotating the rotary switch 13. The valve core can block or avoid a passage between any liquid inlet and the liquid outlet 21, so as to achieve conduction and blocking of the liquid. In this embodiment, the first liquid inlet 11 is configured for an intravenous infusion liquid. It may be understood that the first liquid inlet 11 is connected with an infusion apparatus through an infusion tube, and the liquid stored in the infusion apparatus may be Ringer lactate solution, and sodium, potassium, magnesium, calcium and glucose injection and the like. The second liquid inlet 12 is configured for a syringe liquid, and the liquid outlet 21 is connected with a venipuncture needle. Under the action of the rotary switch 13, the three-way valve 10 has three use states: the first liquid inlet 11 is connected with the liquid outlet 21 while the second liquid inlet 12 is not connected with the liquid outlet 21, corresponding to an intravenous infusion mode; the second liquid inlet 12 is connected with the liquid outlet 21 while the first liquid inlet 11 is not connected with the liquid outlet 21, corresponding to an intravenous injection mode; and both the first liquid inlet 11 and the second liquid inlet 12 are connected with the liquid outlet 21, corresponding to a dosing operation in the case of intravenous infusion. A specific structure of the three-way valve 10 may employ the structural design of an existing medical three-way valve, and thus will not be described in detail here.

The information matching structure includes a first identification code for identifying identity information of the three-way valve 10 and an identifier for identifying the first identification code. The first identification code may be a bar code or a two-dimensional code, on which the identity information corresponding to the three-way valve 10 is stored. The first identification code may be arranged the three-way valve 10 by attaching or code spraying, or attached to a packaging bag/box of the three-way valve 10. In this embodiment, the first identification code is attached to the packaging box of the three-way valve 10 by pasting.

The detection structure includes a first flow sensor 18 for detecting a liquid flow rate at the first liquid inlet 11, a second flow sensor 17 for detecting a liquid flow rate at the second liquid inlet 12 and a pressure sensor 19 for detecting a pressure at the liquid outlet 21.

The controller is capable of acquiring the identity information identified by the identifier and acquiring information detected by the first flow sensor 18, the second flow sensor 17 and the pressure sensor 19, correspondingly recording the information to corresponding identity information items, and controlling the warning indicator 15 to give the alarm when the information is abnormal.

The controller includes a chip 20 and a computer terminal system. A code scanner is electrically or wirelessly connected with the computer terminal system. The identity information acquired by scanning the first identification code by the code scanner is transmitted to a computer mobile terminal. The first flow sensor 18, the second flow sensor 17 and the pressure sensor 19 are connected with the chip 20, and are transferred to the computer mobile terminal by the chip 20. The chip 20 acquires data of the first flow sensor 18, data of the second flow sensor 17 and data of the pressure sensor 19 and transmits the data to the computer mobile terminal, and the computer mobile terminal acquires and records the corresponding data. In the case that the information is abnormal, the computer mobile terminal controls the warning indicator 15 to give an alarm.

It may be understood that a code scanning identification operation is performed before the three-way valve 10 is connected with the infusion apparatus or the syringe. The code scanner is electrically or wirelessly connected with the controller, the code scanner is configured to identify the first identification code, and to transmit the identified identity information to the controller. The controller acquires and records the identity information. Afterwards, the liquid inlet of the three-way valve 10 is connected with the corresponding infusion apparatus or the syringe (the first liquid inlet 11 is connected with an intravenous infusion tube, or the second liquid inlet 12 is connected with the syringe). The liquid outlet 21 is connected with a venipuncture needle to achieve the connection of the infusion system. At this time, the infusion system is in a state waiting for infusion. The pressure sensor 19 can acquire a peripheral venous pressure of a patient, form a pressure signal and transmit the pressure signal to the controller. When the infusion apparatus is turned on or the syringe is configured to inject the liquid, an infusion state is started. The first flow sensor 18 and/or the second flow sensor 17 receive liquid flow rate information and transmit the flow rate signal to the controller. The controller receives the flow rate signal and records the corresponding flow rate information to a corresponding identity information item.

After acquiring the identity information, the controller collects and records the information from the first flow sensor 18, the second flow sensor 17 and the pressure sensor 19 to the identity information, indicating that the information measured by the first flow sensor 18, the second flow sensor 17 and the pressure sensor 19 is the information at the three-way valve 10, so as to achieve information matching.

In the case that the information is abnormal, the warning indicator 15 gives the alarm. The abnormal information includes, but is not limited to, abnormal liquid input and abnormal liquid output and the like.

The abnormal liquid input is as follows: after the first liquid inlet 11 is connected with the intravenous infusion tube or the second liquid inlet 12 is connected with the venipuncture needle, and the liquid outlet 21 is connected with the venipuncture needle, if the controller receives the pressure information but does not receive the liquid flow rate information, it is determined that the liquid input is abnormal, and the warning indicator 15 gives the alarm. The way of giving the alarm may include: broadcasting “please check the liquid” by the voice broadcast, characterizing an abnormal situation by a warning light, or displaying the abnormal situation by a display screen.

The abnormal liquid output includes information mismatching and abnormal connection. The information mismatching is manifested in that after the infusion system is connected, the controller receives the liquid flow information but does not receive the pressure information, it is determined that the drug is injected illegally, and the warning indicator 15 gives the alarm. The way of giving an alarm may include: broadcasting “illegal drug injection, please connect the patient” by the voice broadcast, characterizing the abnormal situation by a warning light, or displaying the abnormal situation by a display screen. The abnormal connection is manifested in that after the infusion system is connected, the controller receives the liquid flow information and the pressure information, but the pressure exceeds a set threshold, it is determined that the puncture needle is inserted abnormally, the waning indicator 15 gives the alarm. The way of giving an alarm may include: broadcasting “please check puncture needle” by the voice broadcast, characterizing the abnormal situation by a warning light, or displaying the abnormal situation by a display screen.

It may be understood that different abnormal situations may be presented by different characterization ways by the warning indicator 15, such as different voice broadcast contents, different colors or flashing times of the warning light, and different contents displayed on the display screen.

In this embodiment, the warning indicator 15 is a voice warning indicator arranged at the three-way valve 10. The warning indicator 15 is integrated on the three-way valve 10, thus facilitating operation staff to rapidly localize and check the infusion system with problems.

From above, the infusion system provided by the present disclosure can accurately manage the drug dosage through the detection structure and the information matching structure, which can replace a manual management method, is convenient for the accurate control of the amount of the in and out of the drugs, and conducive to solving the problem of drug waste.

The infusion system provided by this embodiment can be applied to the management of controlled drugs such as opioids in infusion and injection, and can also be applied to the use of other intravenous infusion drugs, which is not limited here.

In another embodiment of the present disclosure, the three-way valve 10 is provided with an attachment area 14, the attachment area 14 is configured for attaching a second identification code 141, and the second identification code 141 is configured for identifying patient information. The identifier can identify the second identification code 141. The controller can acquire the patient information identified by the identifier, and associate and match the patient information with the identity information of the three-way valve 10.

The second identification code 141 may be a bar code or a two-dimensional code. The patient information is stored in the second identification code 141, including, but not limited to, the patient name, the identity card information, the medical record number, and the inpatient bed number. Combined with a hospital scene, the second identification code 141 is printed by a nurse, and an anesthesiologist identifies the information of the second identification code 141 through the identifier. After checking the patient name and medical record number of the patient, the second identification code 141 is pasted in the attachment area 14. The patient information identified by the identifier is transmitted to the controller, and the controller acquires the patient information and associates and matches the patient information with the identity information of the three-way valve 10.

Therefore, the infusion system can accurately localize the patient information and the injected drug dosage of the patient, facilitate the strict control of the drug, and assist the anesthesiologist to manage an intravenous infusion liquid passage of a surgical patient, and thus has broad clinical prospects.

In another embodiment of the present disclosure, the controller sends successful matching information after receiving the identity information of the three-way valve 10 and the patient information. After the information of the three-way valve 10 is matched with the patient information, the successful matching information is sent out by voice or other means to provide positive feedback to the operation staff/anesthesiologist, so as to guide the operation staff/anesthesiologist to successfully complete the infusion operation.

In another embodiment of the present disclosure, please referring to FIG. 1, the three-way valve 10 is provided with a reader 16 for identifying drug information of a syringe (not shown) at the second liquid inlet 12. A radio frequency tag is attached to a nipple of the syringe. When the syringe is connected with the second liquid inlet 12, the reader 16 identifies the drug information on the radio frequency tag, the drug information includes, but is not limited to, the drug species and the drug concentration and the like, and a drug signal is formed and uploaded to the chip 20. When drug injection is started, the second flow sensor 17 collects drug flow information, including injection time, injection dosage and stop time, and the information is uploaded to the chip 20. The chip 20 receives and processes the drug signal and the drug flow signal to form an injection information signal, which is uploaded to the computer terminal system. The injection information, including but not limited to the drug name, the drug concentration, the injection time, the injection dosage and the stop time, is displayed and recorded by the computer terminal system.

In other embodiments, the syringe is an ordinary syringe without a radio frequency tag. The three-way valve 10 is not provided with the reader 16. The computer terminal system only records the injection time, the injection dosage and the stop time. If the second liquid inlet 12 is connected with a three-way threaded cap (no drug is injected), the injection time, the injection dosage and the stop time are still displayed, and the injection dosage is zero at this time.

In another embodiment of the present disclosure, the detection structure further includes a second pressure sensor (not shown in figure) at the second liquid inlet 12, the second pressure sensor sends a pressure signal when the syringe is connected with the second liquid inlet 12, and the controller sends successful connection information of the syringe when receiving the pressure signal. When the syringe is connected with the second liquid inlet 12, the successful connection information is sent out by voice or other means to provide positive feedback to the operation staff/anesthesiologist, so as to guide the operation staff/anesthesiologist to successfully complete the infusion operation.

In another embodiment of the present disclosure, the controller sends system start information when receiving the flow information of the first liquid inlet 11 or the second liquid inlet 12 and the pressure information of the liquid outlet 21. After the connection of the infusion system is completed, the system start information is sent out by voice or other means to provide positive feedback to the operation staff/anesthesiologist, so as to guide the operation staff/anesthesiologist to successfully complete the infusion operation.

An operation method of the infusion system is as follows.
1. After checking the patient information, the packaging bag of the three-way valve 10 is opened to take out the three-way valve 10, the first liquid inlet 11 of the three-way valve is connected with an infusion apparatus interface, the liquid outlet 21 is connected with the peripheral venipuncture needle interface, and if injection is needed, the second liquid inlet 12 is connected with the syringe to construct a complete intravenous infusion system.
2. A circulating nurse prints the second identification code 141, after the patient name and the medical record number of the patient are checked by the anesthesiologist and the circulating nurse, the second identification code is attached to the attachment area 14 of the three-way valve 10.
3. The anesthesiologist scans the second identification code 141 and the first identification code using the code scanner, after the anesthesiologist manually matches the information of the first identification code with the information of the second identification code in the computer terminal system, or the first identification code and the second identification code 141 are automatically matched by software, “information matching” is broadcasted by the voice broadcaster.
4. When the intravenous infusion liquid is turned on, the first flow sensor 18 can receive the flow rate information of the intravenous infusion liquid and upload the liquid flow rate signal to the chip 20, and the pressure sensor 19 senses a peripheral venous pressure to generate an intravenous pressure signal, and the intravenous pressure signal is uploaded to the chip 20. The chip 20 transmits the flow rate information and the pressure information to the computer mobile terminal. When the computer mobile terminal receives the liquid flow rate signal and the intravenous pressure signal, the voice broadcaster broadcasts “the smart tee has been opened”. With the delivery of the liquid, the computer mobile terminal collects the injection information, including the injection time, the injection dosage, the stop time and the like, and records the drug injection information.
When the computer mobile terminal only receives the liquid flow rate signal, or only receives the intravenous pressure signal, the computer terminal system prompts that “the smart tee is not opened”.
5. When the second liquid inlet 12 is connected with the syringe, the reader 16 identifies the drug information which includes, but is not limited to, the drug species and the drug concentration on the radio frequency tag, and the drug signal is formed and uploaded to the chip 20. When the injection is started, the second flow sensor 17 collects an injection signal, including injection time, the injection dosage and the stop time, and uploads the injection signal to the chip 20. The chip 20 receives and processes the drug signal and the drug flow signal to form the injection information signal, which is uploaded to the computer terminal system. The injection information, including but not limited to the drug name, the drug concentration, the injection time, the injection dosage and the stop time and the like, is displayed and recorded by the computer terminal system.
6. If the second liquid inlet 12 is connected with the ordinary syringe, only the injection time, the injection dosage and the stop time are recorded. If the second liquid inlet 12 is connected with the three-way threaded cap (no drug is injected), the injection time, the injection dosage and the stop time are still displayed, and the injection dosage is zero at this time.
7. After the intravenous infusion system is constructed, the chip 20 does not receive the intravenous pressure signal, and the chip 20 forms the warning signal, and the warning signal is transmitted to the computer terminal system. At this time, the computer terminal system continues to collect the injection information and marks “illegal injection”. Meanwhile, an audio signal is formed by the chip 20, and “illegal injection, please connect the patient” is broadcasted through the voice broadcaster.

Further, other liquid management schemes are provided.
1. After the intravenous infusion system is constructed, the chip 20 receives the intravenous pressure signal, but does not receive the liquid flow rate signal, the chip 20 forms the reminding signal, the reminding signal is transmitted to the voice broadcaster, and the voice broadcaster receives the signal to form the voice broadcast “please check liquid”.
2. After the intravenous infusion system is constructed, the intravenous pressure signal received by the chip 20 exceeds the set threshold, the chip 20 forms the reminding signal, the reminding signal is transmitted to the voice broadcaster, and the voice broadcaster receives the signal to form the voice broadcast “please check puncture needle”.

The above is only the preferred embodiment of the present disclosure, and is not used to limit the present disclosure. Any modification, equivalent substitution, improvement and the like. made within the spirit and principle of the present disclosure should be included in the scope of protection of the present disclosure.

1. An infusion system, comprising:
a three-way valve, provided with a first liquid inlet for an intravenous infusion liquid, a second liquid inlet for a syringe liquid and a liquid outlet communicating with a venipuncture needle;
an information matching structure, comprising a first identification code for identifying identity information of the three-way valve and an identifier for identifying the first identification code;
a detection structure, comprising a first flow sensor for detecting a liquid flow rate at the first liquid inlet, a second flow sensor for detecting a liquid flow rate at the liquid outlet and a pressure sensor for detecting a pressure at the liquid outlet; and
a controller, capable of to acquiring the identity information identified by the identifier and acquiring information detected by the first flow sensor, the second flow sensor and the pressure sensor, correspondingly recording the information to corresponding identity information items and controlling a warning indicator to give an alarm when the information is abnormal.

2. The infusion system according to claim 1, wherein the three-way valve is provided with an attachment area, the attachment area is configured for attaching a second identification code, and the second identification code is configured for identifying patient information, and the identifier is capable of identifying the second identification code.

3. The infusion system according to claim 2, wherein the controller is configured to send successful matching information after receiving the identity information and the patient information.

4. The infusion system according to claim 1, wherein the controller is configured to acquire and record the information detected by the first flow sensor and the second flow sensor, comprising injection time, injection dosage and stop time.

5. The infusion system according to claim 1, wherein the controller is configured to control the warning indicator to give the alarm indicator when the controller receives pressure information of the liquid outlet but not receive flow information of the first liquid inlet or flow information of the second liquid inlet, or when the controller receives the flow information of the first liquid inlet or the flow information of the second liquid inlet but not receive the pressure information of the liquid outlet.

6. The infusion system according to claim 1, wherein the controller is configured to control the warning indicator to give the alarm when a pressure at the liquid outlet is greater than a preset threshold.

7. The infusion system according to claim 1, wherein the warning indicator is a voice warning indicator arranged at the three-way valve.

8. The infusion system according to claim 1, wherein the controller is configured to send system start information when flow information of the first liquid inlet or flow information of the second liquid inlet is matched with pressure information of the liquid outlet.

9. The infusion system according to claim 1, wherein the three-way valve is provided with a reader for identifying drug information of a syringe at the second liquid inlet.

10. The infusion system according to claim 9, wherein the drug information comprises a drug name and a drug concentration.

11. The infusion system according to claim 2, wherein the three-way valve is provided with a reader for identifying drug information of a syringe at the second liquid inlet.

12. The infusion system according to claim 3, wherein the three-way valve is provided with a reader for identifying drug information of a syringe at the second liquid inlet.

13. The infusion system according to claim 4, wherein the three-way valve is provided with a reader for identifying drug information of a syringe at the second liquid inlet.

14. The infusion system according to claim 5, wherein the three-way valve is provided with a reader for identifying drug information of a syringe at the second liquid inlet

15. The infusion system according to claim 6, wherein the three-way valve is provided with a reader for identifying drug information of a syringe at the second liquid inlet.

16. The infusion system according to claim 7, wherein the three-way valve is provided with a reader for identifying drug information of a syringe at the second liquid inlet.

17. The infusion system according to claim 8, wherein the three-way valve is provided with a reader for identifying drug information of a syringe at the second liquid inlet.

18. The infusion system according to claim 11, wherein the drug information comprises a drug name and a drug concentration.

19. The infusion system according to claim 12, wherein the drug information comprises a drug name and a drug concentration.

20. The infusion system according to claim 13, wherein the drug information comprises a drug name and a drug concentration.
